(19) United States
(12) Patent Application Publication
(10) Pub. No.: US 2023/0414469 A1
(43) Pub. Date: Dec. 28, 2023

(54) SUSTAINABLE SHAMPOO

(71) Applicant: JOHNSON & JOHNSON CONSUMER INC., Skillman, NJ (US)

(72) Inventors: Euen Ekman-Gunn, Hopewell, NJ (US); Ana Carolina Nogueira, Jersey City, NJ (US); Daniel Knatz, Sarasota, FL (US)

(21) Appl. No.: 18/211,880

(22) Filed: Jun. 20, 2023

Related U.S. Application Data
(60) Provisional application No. 63/355,307, filed on Jun. 24, 2022.

(51) Int. Cl. A61K 8/46; A61K 8/41; A61K 8/73; A61Q 5/02; A61Q 5/12

(52) U.S. Cl. CPC A61K 8/466; A61K 8/416; A61K 8/732; A61Q 5/02; A61Q 5/12; A61K 2800/87

(57) ABSTRACT

The present invention provides a sustainable shampoo that includes alapha-olefin sulfonate; betaine; sodium hydrolyzed potato starch dodecenylsuccinate; and water. The sustainable shampoo is in the form of a solid structure. The sustainable shampoo is gentle and non-irritating. Recyclable or biodegradable paper packaging can be used preferably to reduce energy consumption. The present invention also provides a method for preparing the sustainable shampoo.

### CROSS REFERENCE RELATED APPLICATION

The present application claims the benefit of the earlier filing date of U.S. patent application 63/355,307, filed Jun. 24, 2022, the entirety of which application is hereby incorporated by reference herein as if fully set forth herein.

### FIELD

The invention relates to the field of cosmetics, in particular to a sustainable shampoo and its preparation method.

### BACKGROUND

At present, there are various shampoo products for hair cleaning but most of them are in the form of liquid. It may be inconvenient for the user to carry and use the liquid shampoo. Increased use of plastic containers causes pollution of the environment. Reducing plastic packaging for products is desirable in the cosmetics and personal care industry.

A number of shampoo products contain ingredients that can elicit irritation, including hair damage and skin irritation upon long term use. For example, silicone oils such as octacyclotetrasiloxane (D4) or decamethylcyclopentasiloxane (D5) have been added to shampoos to enhance smoothness of hair. It has been reported that the European Chemicals Agency (ECHA) found that D4 and D5 are persistent, bioaccumulative, toxic, especially highly persistent and bioaccumulative in the environment, so they are in REACH Annex XIII.

U.S. Pat. No. 4,996,006 to Constantine & Weir discloses solid shampoo composition in compact needle form with water as a binder. The reference discloses that the composition comprises 70% to 90% detergent, preferably sodium lauryl sulphate, in solid needle form and 1% to 10% water.

U.S. Pat. No. 10,933,005 to Henkel AG & Co. KGaA discloses hair treatment agent having anti-washout effect. The reference discloses a hair treatment agent that comprises: at least one anionic surfactant from the group of alpha-olefin sulfonates; at least one anionic surfactant from the group of taurides; at least one amphoteric surfactant; at least one silver salt selected from silver acetate, silver adipate, silver citrate, silver galactarate, silver gluconate, silver lactate, silver malate, silver mandelate, silver salicylate, silver succinate, silver sulfate, and silver tartrate; and at least one organic acid selected from adipic acid, succinic acid, citric acid, acetic acid, galactaric acid, gluconic acid, malic acid, mandelic acid, lactic acid, salicylic acid, sulfuric acid, silver acetate, and tartaric acid, to reduce or prevent washing out of color from dyed hair.

U.S. Pat. No. 10,912,728 to L′Oreal discloses cosmetic composition containing linear alpha-olefin sulfonates, anionic surfactants, and non-ionic and/or amphoteric surfactants, and cosmetic treatment method. The reference discloses that the composition may be in any presentation form, including, e.g., an aqueous, alcoholic or aqueous-alcoholic or oily solution or suspension; a solution or dispersion of the lotion or serum type; an emulsion, an aqueous or anhydrous gel, or any other cosmetic form. The reference also discloses that composition is preferably aqueous and comprises water at a concentration preferably ranging from 30% to 98% by weight, especially from 50% to 95% by weight and better still from 60% to 90% by weight, relative to the total weight of the composition.

U.S. Pat. No. 9,877,907 to Kao Corporation discloses internal olefin sulfonate composition. The reference discloses that the composition comprises (A) an internal olefin sulfonate having 12 carbon atoms and (B) an internal olefin sulfonate having 14 carbon atoms, wherein a content mass ratio of the component (A) to the component (B), (A/B), is from 10/90 to 90/10, and a total content of the component (A) and the component (B) in the internal olefin sulfonate is from 60 to 100% by mass.

U.S. Pat. No. 4,320,033 to Lion Corporation discloses solid detergent composition. The reference discloses that composition contains: (A) 40 to 89.9% by weight of at least one linear alpha-olefin sulfonate having 12 to 28 carbon atoms; (B) 10 to 60% by weight of at least one, tertiary amine oxide having a specified formula, and (C) 0.1 to 20% by weight of water.

U.S. Published Application No. 20200399563 to Ecolab USA, Inc. discloses solidifying non-ionic surfactants. The reference discloses a composition that comprises a liquid nonionic surfactant; and a solid binder comprising a natural polymer, urea, a urea derivative, a polyacrylate, a chelant, a PEG, an inorganic acid and/or salt thereof, an organic salt and/or salt thereof, aromatic sulfonates or a combination thereof; wherein the solid binder and the liquid surfactant are in a ratio of between about 4:1 to about 1:60 by actives; wherein the composition is a solid and the liquid surfactant is solidified in the composition.

U.S. Published Application No. 20190233761 to Ecolab USA, Inc. and BASF SE discloses a solid cleaning composition. The reference discloses that the composition comprises a solidified surfactant blend that comprises; (1) at least one metal alkyl ether sulfate having a specified formula, and (2) a solid surfactant; wherein (1) and (2) are present in a weight ratio of from about 30:70 to about 40:60 based on a total weight of said solidified surfactant blend; and wherein said solidified surfactant blend has a flow function value of less than 0.4 as determined using a Brookfield powder flow tester. The reference discloses that the solid surfactant may be sodium alpha-olefin sulfonate (C14-16).

U.S. Pat. No. 10,912,956 to Mary Kay Inc. discloses cosmetic compositions and uses thereof. The reference discloses that the compositions, which comprise Hylocereus undatus fruit extract and Aloe barbadensis leaf extract, may also contain sodium C14-16 olefin sulfonate.

U.S. Published Application No. 20190365623 to L'Oreal discloses a non-sulfate concentrated rinse-off cleansing composition. The reference discloses that the composition comprises (a) about 25 to about 65 wt. % of a surfactant system comprising: (i) about 10 to about 50 wt. % of one or more non-sulfate anionic surfactants, salts thereof, or a mixture thereof; (ii) about 2 to about 25 wt. % of one or more alkyl polyglucosides; and (iii) about 2 to about 25 wt. % of one or more amphoteric surfactants; and (b) about 0.1 to about 10 wt. % of one or more conditioning agents; and (c) about 35 to about 75 wt. % of water. The reference discloses that the composition may comprise C10-C24 olefin sulfonates.

U.S. Published Application No. 20200129411 to Lonza Ltd. discloses a shampoo bar composition, at least 60 w/w % of one or more surfactants, wherein at least one or more of the surfactant comprises a polyglyceryl ester. In an embodiment, the composition also contains an anionic surfactant, including, e.g., sodium lauroyl methyl isethionate, sodium cocoyl methyl isethionate, sodium oleoyl methyl isethionate, olefin sulphonates, sodium lauryl sulfate, olefin sulphonate, disodium laureth sulfosuccinate, sodium lauryl sulfoacetate, sulfonated methyl esters, sodium cocoyl glutamate, or sodium lauroyl sarcosinate.

WO2022027067 to the Procter & Gamble Company discloses a water-soluble pouch for hair care. The water-soluble pouch contains an internal volume and a plurality of prills within the internal volume. The plurality of prills contains a cationic surfactant selected from behentrimonium methosulfate, brassicamidopropyl dimethylamine, behentrimonium chloride, stearamidopropyl dimethylamine and behenamidopropyl dimethylamine.

A mild, non-irritating, healthy and environmentally friendly sustainable shampoo is desirable.

### SUMMARY

In view of the above, the present invention provides a sustainable shampoo and its method of preparation. The sustainable shampoo is in a solid state and is mild and non-irritating as there are no irritating components such as silicone oil. Recyclable or biodegradable paper packaging for the sustainable shampoo can reduce energy consumption and protect health and the environment. The sustainable shampoo can be used in a solid state, or a user can add water to the sustainable shampoo prior to use.

First, the present invention provides a sustainable shampoo comprising the following components by percentage weight (when in dry form):
about 30% to about 50% alpha-olefin sulfonate, preferably about 35% to about 45% alpha-olefin sulfonate, preferably about 35% to about 40% alpha-olefin sulfonate;
about 20% to about 40% betaine, preferably about 25% to about 35% betaine, preferably about 25% to about 30% betaine, e.g., cocamidopropyl betaine;
about 0.5% to about 5% sodium hydrolyzed potato starch dodecenylsuccinate, preferably about 1.0% to about 4% sodium hydrolyzed potato starch dodecenylsuccinate, preferably about 2.0% to about 3.0% sodium hydrolyzed potato starch dodecenylsuccinate;
about 2% to about 10% water, preferably about 3% to about 5% water.

In the present invention, the alpha-olefin sulfonate has excellent viscosity, hard water stability, detergency, foam characteristics, and pH stability over a broad pH range. It is easy to rinse and biodegradable and is mild to the skin.

NATRASURF ® PS-111, also known as sodium hydrolyzed potato starch dodecenylsuccinate, is a proprietary polymeric surfactant derived from the reaction of food-grade potato starch dextrin with dodecenylsuccinic anhydride, that may be used to improve mildness and foaming attributes of the sustainable shampoo.

Cocamidopropyl betaine may be used as an emulsifier, cleansing agent, and foam booster in the sustainable shampoo.

Additional viscosity increasing agents, including but not limited to, PEG-150 pentaerythrityl tetrastearate and PPG-2 hydroxyethyl cocamide, may be employed.

Salt, including but not limited to, sodium benzoate and sodium citrate, may be used to help the sustainable shampoo fall apart in H2O when reconstituted.

Hair conditioning agents can regulate wet and dry combing properties of hair, reduce electrostatic effect of hair, and enhance suppleness of hair. Examples include, but are not limited to polyquaternium-22 (N,N-dimethyl-N-(2-propenyl)-, chloride, polymer with 2-propenoic acid); and polyquaternium-39 (polymer with N, N-dimethyl-N-2-propenyl-2-propen-1-aminium chloride and 2-propenoic acid). Additional examples include PEG-7 amodimethicone (dimethyl, 3-hydroxypropyl methyl, ethoxylated, 3-bis(2-carboxyethyl)aminopropyl ether).

Additional antistatic agents, including, e.g., linoleamidopropyl PG dimonium chloride phosphate (1-propanaminium, 2,3-dihydroxy-n,n-dimethyl-n-[3-(1-oxo-9,12-octadecadienylamino)propyl]-, 3-phosphate triester, trichloride, can also be used.

Optionally, the sustainable shampoo further comprises essential oils; and/or scalp nutrients; and/or fragrances.

Essential oils, which are fragrant and extracted from natural plants, can reduce nervous tension and cause a pleasant feeling.

Scalp nutrients can provide the desired nutrients to the scalp to maintain the scalp in a healthy state.

Optionally, the pH range of the sustainable shampoo of the invention is about 4.0% to about 5.0%. The sustainable shampoo is generally weakly acidic, conforming to the pH range of healthy hair and scalp so it can protect hair and scalp. Wherein, the pH value of the sustainable shampoo refers to the pH value of an aqueous solution of the sustainable shampoo of the invention.

Alternatively, the packaging material of the sustainable shampoo can be, but is not limited to, paper. The sustainable shampoo of the present invention can be packed as, but is not limited to, a box, the package material of which can be plastic or paper. For the sake of environmental protection and sustainable development, the packaging material of sustainable shampoo is preferred to be paper other than plastic but is not limited to paper. Compared to a plastic case, it can be more cost effective and energy efficient by using paper packaging. Further, compared to the conventional liquid shampoos, the sustainable shampoo can reduce the use of a large amount of packaging material and reduce the transporting capacity. The sustainable shampoo can also reduce usage of water and has a wide range of applications.

The sustainable shampoo proposed by the first aspect of the invention adopts alpha-olefin sulfonate and other raw materials which are mild and non-irritating to skin. The sustainable shampoo is good at foaming, well-adjusted, easy to rinse and excellent in use.

The sustainable shampoo is suitable for all kinds of people and can widely be used for dry and oily hair. It is a healthy and mild shampoo and skin care product without soap base, mineral oil and silicone oil.

Secondly, a method for preparing a sustainable shampoo proposed by the first aspect of the invention is as below:
Weigh each component according to the proportion of the formula, put it into a stirring pot, and obtain a mixture after stirring;
Then the mixture is molded to obtain a solid sustainable shampoo.

Regarding the sustainable shampoo proposed by the second aspect of the invention, the process of the preparation method is simple.

For the sake of environmental protection and sustainable development, the sustainable shampoo of the invention adopts packaging materials as paper other than plastic, but not limited to paper. For example, the sustainable shampoo is packaged with recyclable or biodegradable paper, so as to reduce the plastic packaging of cleaning and care products thereby reducing the environment pollution and damage and achieving sustainable development. Further, the sustainable shampoo mentioned in the invention also has good biodegradability. Together with the biodegradable paper packaging, the sustainable shampoo of the invention can be energy saving, environmentally friendly and at reduced production cost.

The shape and size of the sustainable shampoo can be varied by presetting various shapes. For example, the cross section shape of the sustainable shampoo can be a cylinder, circle, rectangle, triangle or polygon.

The advantages of the invention will be explained in part of the following specification, part of which is clear according to the specification or can be realized through the embodiment of the invention.

### BRIEF DESCRIPTION OF THE DRAWINGS

In order to explain the content of the invention more clearly, the following shows the detail in combination with attached drawings and specific embodiment.

FIG. 1 is a picture showing an embodiment of the invention.

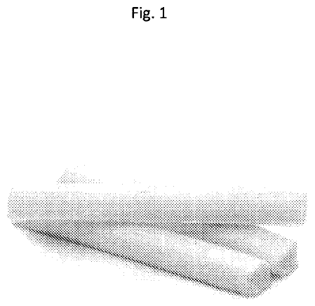

FIG. 2 is a picture showing use of the invention, wherein the sustainable shampoo is reconstituted in water.

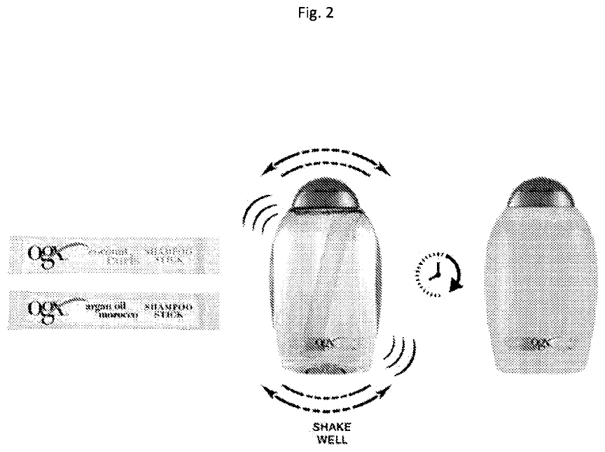

### DETAILED DESCRIPTION OF THE EMBODIMENTS

The following is the preferred embodiment of the invention. It should be pointed out that for technicians in the field, on the premise of not deviating the invention from the principle of embodiment, some improvements and retouching can be made, and these improvements and retouching are also regarded as the protection scope of the embodiment of the invention.

Embodiment of the present invention is further described as the following. Embodiment of the invention is not limited to the following specific embodiment. Without amendment in the claims, changes can be implemented appropriately. Unless there is any special instruction, the raw materials and other chemical reagents used in the embodiment of the present invention are all commercially available.

Various combinations and amounts of ingredients were investigated until the sustainable shampoo had the right balance of acceptable characteristics, including:
a. Dissolution
b. Hardness
c. Mildness

The preparation steps for the sustainable shampoo may be as below:
Each component is weighed according to the proportion of the formula and mixed evenly to obtain a mixture.
The mixture is shaped into the required shape to obtain the sustainable shampoo.

### EXAMPLE

A sustainable shampoo having the ingredients in Table 1 was prepared in accordance with the method below.

TABLE 1

| | | | Percent |
|---|---|---|---|
| | | Percent | (recon- |
| Tradename | INCI | (dry) | stituted) |
| Water | Water | QS | 78.63 |
| Rhodacal A 246 PF | Sodium C14-16 Olefin | 38.32 | 8.19 |
| | Sulfonate | | |
| Tego Betain CK D MB | Cocamidopropyl Betaine | 29.48 | 6.30 |
| (Powder Betain) | | | |
| Structure PS-111 | Sodium Hydrolyzed | 2.34 | 0.50 |
| | Potato Starch | | |
| | Dodecenylsuccinate | | |
| Merquat 280NP | Polyquaternium-22 | 6.08 | 1.30 |
| Merquat 3940 | Polyquaternium-39 | 0.94 | 0.20 |
| Arlasilk EFA-LQ-(AP) | Linoleamidopropyl PG | 3.51 | 0.75 |
| | Dimonium Chloride | | |
| | Phosphate | | |
| Silsense A21 | PEG-7 Amodimethicone | 2.34 | 0.50 |
| Versathix LQ | PEG-150 Pentaerythrityl | 4.63 | 0.99 |
| | Tetrastearate (and) PPG- | | |
| | 2 Hydroxyethyl Cocamide | | |
| | (and) Water | | |
| Fragrance (Kukui) | Fragrance | 3.51 | 0.75 |
| Sodium Benzoate | Sodium benzoate | 2.34 | 0.50 |
| Sodium Citrate | Sodium citrate | 2.34 | 0.50 |
| Citric Acid | Citric Acid 50% solution | 4.16 | 0.89 |
| | (pH adjuster) | | |

Instructions:
a. Mix all powder ingredients in beaker
b. Add liquid ingredients, hand mixing between addition
c. Add Water until ideal consistency is reached

Reconstitution
a. Add known amount of solid product to bottle/jar
b. Add 3.68× mass of water to bottle/jar and shake
c. Let rest until product is full dissolved
d. (Optional) Shake final solution

The sustainable shampoo can be assessed as follows:
Assess user experience.
   Compared to solid shampoo on the market.
   Compared to liquid shampoo on the market.
Assess properties:
   Foam
   Dry combing
   Wet combing
   Moisturizing
   Rinsing
   Degree of cleanliness

It is expected that sustainable shampoo of the invention is superior to other shampoos on the market. It is also expected that consumers preference of the sustainable shampoo will be higher than other products on the market.

The sustainable shampoo of the invention can be packaged with recyclable or biodegradable cardboard or paper, so as to reduce the use of plastics. In order to protect the environment, limiting the use of plastic and not polluting the environment can achieve sustainable development. The invention embodies the concept of environmental protection and sustainable development and has great application prospects. The sustainable shampoo provided by the embodiment of the invention is mild, non-irritating, healthy and safe, with high durability, and can be used repeatedly or as a unit quantity. Compared with the existing shampoo products, the sustainable shampoo of the invention is more economical and practical, which is of great significance for prevalence in use of the sustainable shampoo.

The above disclosure is only a better embodiment of the invention, and of course cannot be used to limit the scope of the rights of the invention. Technicians in the field can understand all or part of the process of the above embodiment, and the equivalent changes made according to the claims of the invention still fall within the scope of the invention.

1. A sustainable shampoo, wherein the sustainable shampoo comprises the following components:
about 30% to about 50% alpha-olefin sulfonate;
about 20% to about 40% betaine;
about 0.5% to about 5% sodium hydrolyzed potato starch dodecenylsuccinate; and
about 2% to about 10% water.

2. The sustainable shampoo according to claim 1, wherein the betaine is cocamidopropyl betaine.

3. The sustainable shampoo according to claim 1, further comprising one or more hair conditioning agents.

4. The sustainable shampoo according to claim 3, wherein the one or more hair conditioning agents is selected from the group consisting of polyquaternium-22 (N,N-dimethyl-N-(2-propenyl)-, chloride, polymer with 2-propenoic acid); polyquaternium-39 (polymer with N, N-dimethyl-N-2-propenyl-2-propen-1-aminium chloride and 2-propenoic acid); and PEG-7 amodimethicone (dimethyl, 3-hydroxypropyl methyl, ethoxylated, 3-bis(2-carboxyethyl) aminopropyl ether).

5. The sustainable shampoo according to claim 1, further comprising one or more additional viscosity increasing agents.

6. The sustainable shampoo according to claim 5, wherein the additional viscosity increasing agents is selected from the group consisting of PEG-150 pentaerythrityl tetrastearate and PPG-2 hydroxyethyl cocamide.

7. The sustainable shampoo according to claim 1, further comprising one or more salts.

8. The sustainable shampoo according to claim 7, wherein the one or more salts are selected from the group consisting of sodium benzoate and sodium citrate.

9. The sustainable shampoo according to claim 1, further comprising one or more ingredients selected from essential oils; scalp nutrients; and fragrances.

10. The sustainable shampoo according to claim 1, wherein the pH range of the sustainable shampoo is 4.0 to 5.0.

11. The sustainable shampoo according to claim 1, wherein the sustainable shampoo uses recyclable or biodegradable paper packaging.
